(19) United States
(12) Patent Application Publication
(10) Pub. No.: US 2025/0161270 A1
(43) Pub. Date: May 22, 2025

(54) PHARMACEUTICAL COMPOSITION AND MEDICAMENT COMPRISING L-TRYPTOPHAN, L-5-HYDROXYTRYPTOPHAN AND A PERIPHERAL DEGRADATION INHIBITOR

(71) Applicant: JOACHIM KAMPRAD, RHEINE (DE)

(72) Inventor: JOACHIM KAMPRAD, RHEINE (DE)

(21) Appl. No.: 18/839,100

(22) PCT Filed: Feb. 8, 2023
(86) PCT No.: PCT/EP2023/053129

§ 371 Date: Aug. 16, 2024

(30) Foreign Application Priority Data
Feb. 16, 2022 (DE) 10 2022 103 658.8

(51) Int. Cl. A61K 31/405; A61K 31/165; A61K 31/198; A61P 25/04

(52) U.S. Cl. CPC A61K 31/405; A61K 31/165; A61K 31/198; A61P 25/04

(57) ABSTRACT

The present invention relates to the simultaneous use of L-tryptophan (L-Try) and L-5-hydroxytryptophan (5-HTP) and a peripheral degradation inhibitor for the prevention and therapy of pain, depression, sleeping disorders and other serotonin-dependent diseases or disorders of the CNS. All the components of the medicament are supposed to be released together. Carbidopa and/or benserazide are to be used as said peripheral degradation inhibitors for the tryptophan and the 5-hydroxytryptophan.

The invention relates to a pharmaceutical composition or medicament comprising L-tryptophan and L-5-hydroxytryptophan and a peripheral degradation inhibitor. In particular, the present invention relates to the simultaneous use of L-tryptophan, L-5-hydroxytryptophan and at least one peripheral degradation inhibitor for the prevention and therapy of pain, depression, sleeping disorders and other serotonin-dependent diseases or disorders of the CNS.

### BACKGROUND OF THE INVENTION

L-Tryptophan (L-Try) and/or L-5-hydroxytryptophan (5-HTP) are used for treating pain, depression, and sleeping disorders, with varying success. L-Tryptophan and/or L-5-hydroxytryptophan is administered in excess in order to thereby enhance the amount of serotonin in the CNS. However, attempts to employ L-Tryptophan or L-5-hydroxytryptophan alone as an effective medicament are not convincing. Despite in part high dosages, the activity is uncertain (high peripheral degradation/competition at the blood-brain barrier/enzyme induction, peripheral), and/or significant side effects occur (including from high serotonin increase/storage even in non-serotonergic neurons). Dietetic experiments were performed with elimination of neutral amino acids competing at the blood-brain barrier (competitive displacement).

The processing and transmission of information in the central nervous system (CNS) take place on the basis of neurochemical transmission. The necessary messengers (neurotransmitters) are synthesized from food ingredients, usually amino acids, and are then available for the corresponding neuronal structures. Many diseases of the CNS are either based on a deficiency in one or more neurotransmitters in the CNS, or are the result of the lacking or defective bioavailability of neurotransmitters. Examples of such messengers include serotonin and dopamine.

Serotonin is widespread in nature and is found in mammals in a relatively high concentration in the CNS (hypothalamus, periaqueductal gray, substantia grisea centralis, limbic system), in the lung, and in the cells having argentaffinity of the digestive tract.

Serotonin has a peripheral effect, inter alia, on the smooth muscle of the vessels of the respiratory and gastro-intestinal tracts. Serotonin displays a particularly significant effect on the central nervous system. Here, it participates in pain control, mood control, sleep regulation, and other serotonin-dependent functions. Dopamine is a catecholamine that occurs, inter alia, in the brain, adrenal glands, and sympathetic nerve endings, and is a neurotransmitter of the hypophyseotropic zones of the hypothalamus. In Parkinson's disease, the concentration of dopamine in reduced in the nuclei of the extrapyramidal system.

Since neurotransmitters, such as serotonin and dopamine, cannot be supplied directly to the CNS because of a lacking mobility through the blood-brain barrier, or significant side effects, biochemical precursors are employed.

The precursor L-DOPA is frequently administered in the therapy of Parkinson's disease, in order to compensate for a systemic deficiency in dopamine in the CNS, especially in the basal ganglia. However, L-DOPA is decomposed to a high extent already peripherally, i.e., in the blood, and in the gastro-intestinal tract, but also at the blood-brain barrier (BBB), into the neurotransmitter, which is desired in principle, and thus does not reach the CNS, or only so in insufficient amounts. Since dopamine as such is not mobile through the blood-brain barrier, it flushes the periphery, but virtually does not enter the brain. This results in the known peripheral side effects, such as nausea, vomiting, cardiovascular disorders, changes in blood pressure, etc. In order to reduce such side effects and increase the amount of L-DOPA available in the CNS, L-DOPA is combined with peripheral degradation inhibitors, since L-DOPA, like L-tryptophan, is degraded peripherally by amino acid decarboxylase. As a consequence, L-DOPA becomes enriched in the plasma and can overcome the blood-brain barrier in a sufficiently high amount. There, L-DOPA is degraded to dopamine as desired. The peripheral degradation pathway of L-DOPA is partially identical with the peripheral degradation pathway of L-tryptophan.

The initial precursor for the neurotransmitter serotonin is L-tryptophan, which is contained in most proteins at 1 to 2%. L-tryptophan occurs in the natural food of humans and is an essential amino acid. Different degradation pathways of L-tryptophan are known. The degradation of L-tryptophan in the liver through 2-3-dioxygenase and through kynureninase is most important quantitatively, with more than 90%. In addition, there is the peripheral degradation of L-tryptophan through L-5-hydroxytryptophan (5-HTP) after decarboxylation into 5-hydroxytryptamine (5-HT=serotonin).

The peripheral degradation of L-tryptophan outside the CNS leads to accumulation of serotonin in the wrong place and thus undesirable side effects, also peripherally, such as blood pressure crises, chronic diarrhea, bronchospasm, cardiac disorders, gastrointestinal disorders, etc. Only a small amount of L-tryptophan escapes the peripheral degradation and can get into the CNS unhindered, where it can be degraded to the desired neurotransmitter. Attempts to administer as large as possible amounts of L-tryptophan in order to achieve an effective accumulation of this amino acid fail because of the side effects that will then occur and because of an increased intracerebral and extracerebral degradation of serotonin and L-tryptophan.

In contrast, L-5-hydroxytryptophan overcomes the blood-brain barrier more quickly (direct precursor) and is thus more difficult in application and has more side effects, especially in combination with a peripheral degradation inhibitor.

The treatment of Parkinson patients with L-DOPA preparations in combination with the peripheral amino acid decarboxylase inhibitors benserazide and carbidopa, or entacapone as an O-methyltransferase inhibitor (COMT-inhibitor) is known.

The combination of L-DOPA with the specific decarboxylase inhibitor benserazide together with a hydrocolloid and some conventional excipients as a sustained release preparation is described in DE 3232873 A1. The relatively quick degradation of the active ingredients in blood has an unfavorable effect.

The combination of L-tryptophan with a specific decarboxylase inhibitor such as benserazide is described in EP 344158 B1 and the combination of 5-HTP with a specific decarboxylase inhibitor such as carbidopa is described in WO 9107960 A1. In order to ensure a permanent offer of L-tryptophan at the blood-brain barrier, the administration of L-tryptophan is indicated either at high concentrations, or in very short intervals, or in a sustained release form. From the U.S. patent specifications 4 126 672, 4 140 755, 4 167 558 and the above-mentioned DE 3232873 A1, formulations showing a sustained release upon oral application have been known. These are capsules or tablets that are so balanced hydrodynamically to possess a specific density of less than 1, and will float in gastric juice with a specific density of from 1.0004 to 1.010. The sustained drug release of these formulation is based on a mixture of the active ingredients with one or more hydrophilic hydrocolloids.

The combination of L-tryptophan with a peripheral degradation inhibitor, such as benserazide and carbidopa, in a sustained release form for the treatment of pain is described in EP 0 344 158 B1. Since the relatively quick degradation of benserazide and carbidopa in blood plasma (short plasma half-lives) is known, a sustained release of both L-tryptophan and the peripheral degradation inhibitor in a particular dosage form is described. In this, the permanent offer of L-tryptophan at the blood-brain barrier (in a sustained release form) is supported by a permanent offer of a peripheral degradation inhibitor (benserazide or carbidopa, also in sustained release form).

In expectation of less side effects, improvement of tolerability and simplified galenics, a sustained release of peripheral degradation inhibitors (benserazide and carbidopa) was dispensed with in EP 1 784 177 B1, so that the disclosure relates to the still sustained release of L-tryptophan, and the no longer sustained release of benserazide and carbidopa.

The use of L-tryptophan or L-5-hydroxytryptophan in combination with benserazide and the related low dosages of L-tryptophan or L-5-hydroxytryptophan is described by F. Sicuteri in “Advances in Pain Research and Therapy” Vol. 1, 1976. These combinations have severe drawbacks in terms of their galenics, and in particular the combination of L-tryptophan with benserazide shows a significant delay of the onset of its anti-pain effect of at least two weeks. The administration of L-tryptophan, but also of L-5-hydroxytryptophan (a metabolite of L-tryptophan) increases the central serotonin metabolism, and the serotonin concentration in the CNS. An increased serotonin concentration can be employed in therapy or prevention, especially for pain, depression, sleeping disorders and other serotonin-dependent diseases of the CNS. L-Tryptophan is a physiological compound (essential amino acid) that can be employed for the treatment of, for example, sleeping disorders, depression, pain and other serotonin-dependent complaints or diseases.

### SUMMARY OF THE INVENTION

The present invention relates to a pharmaceutical composition or medicament for the prevention and therapy of pain, depression, sleeping disorders and other serotonin-dependent diseases or disorders of the CNS, containing L-tryptophan (L-Try), L-5-hydroxytryptophan (5-HTP), and at least one peripheral degradation inhibitor, and which composition or medicaments provides provides for a much earlier onset of the anti-pain effect of about 5 days.

Based on the inventor's finding, an essential feature of the invention is to employ another active ingredient in the form of L-5-hydroxytryptophan, in addition to tryptophan. L-5-Hydroxytryptophan is a metabolite of L-tryptophan and the direct precursor of serotonin. In contrast to L-tryptophan, L-5-hydroxytryptophan can overcome the blood-brain barrier (BBB) quickly, does not compete with other neutral amino acids at the BBB, but is more difficult to handle because of more severe side effects, especially in combination with peripheral degradation inhibitors, such as benserazide or carbidopa. L-Tryptophan and L-5-hydroxytryptophan as such have long been known, and also have been used as drugs in humans. Also, the use of benserazide and carbidopa in combination with L-tryptophan or L-5-hydroxytryptophan has been known. It is also known that L-tryptophan and L-5-hydroxytryptophan act at different speeds because of different uptake mechanisms into the CNS, with different side effects and risks. L-Tryptophan is taken up into the CNS more slowly, acts more slowly, but in a sustained mode, and more safely, also in combination with peripheral degradation inhibitors. L-5-Hydroxytryptophan is taken up into the CNS more quickly, acts more quickly, is more potent, but also more risky, and more difficult to handle in combination with peripheral degradation inhibitors. However, it was unexpected that a combination of L-tryptophan, L-5-hydroxytryptophan, and degradation inhibitor were provide for a much earlier onset of the anti-pain effect than a composition without the L-5-hydroxytryptophan. Accordingly, the present invention provides
(1) A pharmaceutical composition or medicament comprising L-tryptophan or a pharmaceutically acceptable salt thereof, L-5-hydroxytryptophan or a pharmaceutically acceptable salt thereof, and at least one peripheral degradation inhibitor, which pharmaceutical composition or medicament is particularly suitable for use the prevention and therapy of pain, depression, sleeping disorders and other serotonin-dependent diseases or disorders of the CNS;
(2) a composition comprising L-tryptophan or a pharmaceutically acceptable salt thereof, L-5-hydroxytryptophan or a pharmaceutically acceptable salt thereof, and at least one peripheral degradation inhibitor for use in the prevention and therapy of pain, depression, sleeping disorders and other serotonin-dependent diseases or disorders of the CNS; or
(3) a method for the prevention and therapy of pain, depression, sleeping disorders and other serotonin-dependent diseases or disorders of the CNS in a subject, which method comprises administering to the subject in need of such treatment a composition comprising L-tryptophan or a pharmaceutically acceptable salt thereof, L-5-hydroxytryptophan or a pharmaceutically acceptable salt thereof, and at least one peripheral degradation inhibitor.

The (pharmaceutical) composition, medicament or method of the invention defined herein before is suitable for the simultaneous intake of L-tryptophan and L-5-hydroxytryptophan in combination with at least one peripheral degradation inhibitor, such as benserazide or carbidopa, for the treatment of pain, depression, sleeping disorders, or other serotonin-dependent diseases of the CNS, preferably in a sustained release form.

Because of the simultaneous use of L-tryptophan and L-5-hydroxytryptophan in combination with, for example, benserazide or carbidopa, and because of the different metabolic mechanisms of the different components with their advantages and disadvantages, an enhanced effectiveness and little side effects are to be considered as compared to the previously employed dosage forms. In addition, an earlier onset of activity occurs because of the simultaneous use of L-5-hydroxytryptophan.

### DETAILED DESCRIPTION OF THE INVENTION

In the (pharmaceutical) composition, medicament or method of the invention defined herein before, the weight ratio of L-tryptophan to L-5-hydroxytryptophan can be set at almost any value. In the following, when referring to L-tryptophan and L-5-hydroxytryptophan, this includes the respective pharmaceutically acceptable salts of said compounds, such as the sodium salts. However, particularly preferred according to the present invention is a (pharmaceutical) composition or medicament in which the weight ratio of L-tryptophan to L-5-hydroxytryptophan is from 20:1 to 1:1. All the components of the (pharmaceutical) composition or medicament according to the invention are supposed to be released together if possible. In particular, carbidopa and/or benserazide are to be used as said peripheral degradation inhibitors for tryptophan and L-5-hydroxytryptophan. The combination of L-tryptophan in a sustained release form (retarded formulation) with a peripheral degradation inhibitor of tryptophan is of high importance to the treatment of the mentioned diseases because of the uptake mechanism through the blood-brain barrier. Preferably, amino acid decarboxylase inhibitors and/or kynureninase inhibitors and/or tryptophan-2-3-dioxygenase inhibitors are used as peripheral degradation inhibitors for L-tryptophan in the present invention. Benserazide and carbidopa are more preferably employed.

It is particularly preferred according to the present invention for the (pharmaceutical) composition or medicament to include L-tryptophan in a sustained release dosage form. Sustained release dosage form for L-tryptophan are per se known to those skilled in the art. The sustained release of L-5-hydroxytryptophan is possible in the same way.

Alternatively or additionally, a (pharmaceutical) composition or medicament according to the invention may also contain L-5-hydroxytryptophan in a sustained release dosage form. Accordingly, a particularly preferred (pharmaceutical) composition or medicament includes L-tryptophan in a sustained release dosage form and L-5-hydroxytryptophan in a sustained release dosage form.

It is particularly preferred according to the present invention for a (pharmaceutical) composition or medicament to include a weight ratio of tryptophan and L-5-hydroxytryptophan to the peripheral degradation inhibitor of 20:1 to 1:1, especially from 3:1 to 5:1.

In (pharmaceutical) compositions or medicaments according to the invention, said peripheral degradation inhibitor is preferably selected from (25)-3-(3,4-dihydroxyphenyl)-2-hydrazino-2-methylpropionic acid (carbidopa) or DL-serine-2-(2,3,4-trihydoxybenzyl) hydrazide hydrochloride (benserazide), and mixtures thereof. Accordingly, a (pharmaceutical) composition or medicament according to the invention containing carbidopa and benserazide is particularly preferred.

The (pharmaceutical) compositions or medicaments according to the invention may further comprise, depending on its dosage form, one or more pharmaceutically acceptable carriers, binders, disintegrants, solvents, flavors, masking agents, coloring agents, etc.

The peripheral degradation inhibitor or inhibitors may also be contained in a non-sustained release dosage form, or in a sustained release dosage form.

The galenic dosage form can be realized in almost any form, for example, in the form of a capsule, tablet, solution, or inhalant.

Said peripheral degradation inhibitor may be administered simultaneously with L-tryptophan.

The present invention is further described in the following Examples, which are however, not to be construed as limiting the invention.

### EXAMPLES

### Example 1 (Comparative Example)

Two studies, not according to the invention, with sustained release L-tryptophan and sustained release benserazide were performed.

1st study: In the Weserland-Klinik hospital in Bad Seebruch/Vlotho, Germany. Title: Analgetic with sustained release in patients with fibromyalgia (L-tryptophan in combination with a peripheral degradation inhibitor, here benserazide), 2004.

22 subjects (12 verum, 10 placebo), double-blind, randomized.

Result: Onset of effect after 2 weeks, pain reduction of about 45% after 4 weeks.

2nd study: Outpatient Pain Department of the Jacobi Krankenhaus hospital in Rheine, Germany. Title: Treatment of chronic pain with a combination drug consisting of L-tryptophan and benserazide (sustained release), 2001.

36 subjects (18 verum, 18 placebo), double-blind, randomized.

Result: Onset of effect after 2 weeks, pain reduction of about 45% after 4 weeks. Another small examination was performed with only 3 doses of benserazide and 5 subjects, in order to model a non-sustained release intake of benserazide. The results were similar.

Result: The studies showed that, in addition to an excellent anti-pain effect that could be attributed to L-tryptophan, the continuous administration (corresponding to a sustained release administration) of L-tryptophan is required for an optimum effect and a constantly high plasma level. In addition, the studies showed that a sustained release administration of benserazide is useful, but not mandatory, to achieve an effect.

As a disadvantage, it was found that the onset of effect unfortunately was surprisingly late, i.e., only after about 2 weeks.

### Example 2

Formulation:
(a) 1 capsule/tablet: 600 mg of L-tryptophan and 62.5 mg of benserazide, retarded, and 50 mg of L-5-hydroxytryptophan, not retarded.

One to 2 capsules/tablets each in the morning and in the evening.
(b) 1 capsule/tablet: 600 mg of L-tryptophan and 62.5 mg of benserazide, retarded, and 50 mg of L-5-hydroxytryptophan, retarded.

Study: It was shown that one to two capsules/tablets each in the morning and in the evening provide for an earlier onset of the anti-pain effect after about 5 days and a pain reduction about 45% after 2 weeks for both formulations (a) and (b), i.e., irrespective of the retardation applied to the L-5-hydroxytryptophan.

1-15. (canceled)

16. A pharmaceutical composition or medicament comprising L-tryptophan or a pharmaceutically acceptable salt thereof, L-5-hydroxytryptophan or a pharmaceutically acceptable salt thereof, and at least one peripheral degradation inhibitor, which contains the L-tryptophan or the pharmaceutically acceptable salt thereof in a sustained release dosage form.

17. The pharmaceutical composition or medicament according to claim 16, wherein the weight ratio of L-tryptophan to L-5-hydroxytryptophan is from 20:1 to 1:1.

18. The pharmaceutical composition or medicament according to claim 16, which further contains the L-5-hydroxytryptophan or the pharmaceutically acceptable salt thereof in a sustained release dosage form.

19. The pharmaceutical composition or medicament according to claim 16, comprising L-tryptophan in a sustained release dosage form and L-5-hydroxytryptophan in a non-sustained release dosage form.

20. The pharmaceutical composition or medicament according to claim 16, wherein the weight ratio of the total of tryptophan and L-5-hydroxytryptophan to said peripheral degradation inhibitors is from 20:1 to 1:1.

21. The pharmaceutical composition or medicament according to claim 16, wherein said peripheral degradation inhibitor is selected from the group consisting of (2S)-3-(3,4-dihydroxyphenyl)-2-hydrazino-2-methylpropionic acid (carbidopa), DL-serine-2-(2,3,4-trihydoxybenzyl)hydrazide hydrochloride (benserazide), and mixtures thereof.

22. The pharmaceutical composition or medicament according to claim 21, which comprises carbidopa and benserazide.

23. The pharmaceutical composition or medicament according to claim 21, which comprises carbidopa or benserazide.

24. The pharmaceutical composition or medicament according to claim 16, which contains at least one of said peripheral degradation inhibitors in a non-sustained release dosage form.

25. The pharmaceutical composition or medicament according to claim 16, which contains at least one of said peripheral degradation inhibitors in a sustained release dosage form.

26. The pharmaceutical composition or medicament according to claim 18, which contains the peripheral degradation inhibitors carbidopa or benserazide in a non-sustained release dosage form.

27. The pharmaceutical composition or medicament according to claim 18, which further contains the peripheral degradation inhibitors carbidopa or benserazide in a sustained release dosage form.

28. The pharmaceutical composition or medicament according to claim 16, which further comprises one or more pharmaceutically acceptable carriers, binders, disintegrants, solvents, flavors, masking agents, and/or coloring agents.

29. The pharmaceutical composition or medicament according to claim 16, which is in the form of a capsule, tablet, solution, or inhalant.

30. The pharmaceutical composition or medicament according to claim 16, which is suitable for the simultaneous intake of L-tryptophan, L-5-hydroxytryptophan and said least one peripheral degradation inhibitor.

31. The pharmaceutical composition or medicament according to claim 27, which is suitable for the simultaneous intake of L-tryptophan, L-5-hydroxytryptophan and the peripheral degradation inhibitors carbidopa or benserazide.

32. The pharmaceutical composition or medicament according to claim 16, which is for the prevention and therapy of pain, depression, sleeping disorders and other serotonin-dependent diseases or disorders of the central nervous system.

33. A method for the prevention and therapy of pain, depression, sleeping disorders and other serotonin-dependent diseases or disorders of the central nervous system in a subject, which method comprises administering to the subject in need of such treatment a composition comprising L-tryptophan or a pharmaceutically acceptable salt thereof, L-5-hydroxytryptophan or a pharmaceutically acceptable salt thereof, and at least one peripheral degradation inhibitor, wherein the composition contains the L-tryptophan or the pharmaceutically acceptable salt thereof in a sustained release dosage form.

34. The method according to claim 33, wherein in the composition the weight ratio of L-tryptophan to L-5-hydroxytryptophan is from 20:1 to 1:1.

35. The method according to claim 33, wherein the composition further contains the L-5-hydroxytryptophan or the pharmaceutically acceptable salt thereof in a sustained release dosage form.
